(19) United States
(12) Patent Application Publication
(10) Pub. No.: US 2003/0092968 A1
(43) Pub. Date: May 15, 2003

(54) Stabilizing device for performing surgical procedures on cardiac tissue

(76) Inventor: Tracey E. Knapp, Lawrenceville, GA (US)

Correspondence Address: GENZYME CORPORATION, LEGAL DEPARTMENT, 15 PLEASANT ST CONNECTOR, FRAMINGHAM, MA, 01701-9322, US

(21) Appl. No.: 10/320,051

(22) Filed: Dec. 16, 2002

Related U.S. Application Data
(63) Continuation of application No. 09/765,514, filed on Jan. 19, 2001, now abandoned.

(51) Int. Cl. A61B 1/32

(52) U.S. Cl. 600/213

(57) ABSTRACT

The present invention relates to a stabilizer that immobilizes tissue at a surgical site. A preferred embodiment of this device is used during cardiac bypass surgery and includes a preferably rectangular, oblong or similar shaped device having a width dimension that is greater than the lengthwise dimension such that an aperture area is formed along three sides of the surgical site so that a graft or anastomosis that is formed in the aperture area allows the stabilizer to be removed without disturbing the graft and the device may be used in combination with flexible tapes or threads to apply an upwardly directed force to the tissue adjacent to the surgical site.

### BACKGROUND OF THE INVENTION

Numerous devices have been used to position tissue at a surgical site to aid in the performing of surgical procedures. Retractors, for example, have been used for many years to hold an artery in position during operations adjacent to the heart to prevent movement of the artery. This serves to minimize the risk of injury to the artery and adjacent tissue and can facilitate the desired anastomosis.

A recently developed procedure, referred to as the minimally invasive direct coronary artery bypass procedure, has been used to graft onto a coronary artery without cardiopulmonary bypass. This procedure involves the grafting of the left internal mammary artery (LIMA) onto the left anterior descending (LAD) or other artery. As this procedure does not require the use of a heart lung machine to oxygenate and pump blood, the morbidity and mortality associated with this procedure is substantially lower than previous bypass techniques. A problem associated with the less invasive procedures, however, is that while the heart continues to pump during the procedure, the motion of the heart can interfere with the surgeon's task of attaching the LIMA to the LAD. There is also a need to stop blood flow in the area of the graft to maintain a clear field of view and provide precise suture placement.

Two basic strategies have been employed to address the problem of operating on a moving site, one being the use of pharmacological agents to limit heart motion, and the other being mechanical, such as a two prong retractor that is pushed down against the heart on both sides of the artery, or alternatively, upward traction away from the moving heart by suction, traction tape or suture thread. Both of these options, however, have problems associated with them. Both options are susceptible to some movement of the vessel graft site or tissue trauma. The use of pharmacological agents is undesirable and may impair circulatory function. Traction by compression of the heart requires an increased amount of downward force on the tissue of the heart along a relatively large surface area. Although this type of device does serve to immobilize the tissue at the surgical site, it may also compromise the ability of the heart to maintain circulation and result in hypotension. Upward traction through the use of suction requires that the entire surface of the device be in contact with the tissue of the heart along a relatively large surface area to maintain suction. As with the compression type of devices, the suction type of device may cause injury, stenosis or occlusion of the vessel when upward traction that is sufficient to immobilize the tissue along the surgical site is used. Additionally, because various surfaces of the heart need to be accessed, it is not always possible or convenient to apply compression or upward traction to each surface of the heart.

There is a continuing need for improvement in devices and methods for retaining tissue at surgical sites to further reduce the risks associated with surgical procedures where the devices and methods are inexpensive, versatile, safe and reliable. The increased use of the above-described mechanical devices have also illustrated the need for a device that provides the desired local stabilization while allowing the surgeon to quickly set up and remove the stabilizing device while also providing access to multiple locations and surfaces on the heart of the patient.

### SUMMARY OF THE INVENTION

The present invention relates to a cardiac stabilizer for immobilizing tissue at a surgical site and to a method of using the stabilizer during a surgical procedure. A preferred embodiment of the stabilizer includes a generally rectangular, oblong or similar shaped retaining element or platform having an aperture area that exposes the surgical site and a holder that is used to position tissue at the surgical site relative to the retaining element. A handle can be attached to or fabricated with the platform so that the user can manipulate the position of the stabilizer as needed. As used herein, the stabilizer generally refers to a device that is movable into a contacting relationship with the tissue of a patient.

In a preferred embodiment of the invention, a connector such as elastic tape or thread is used to position tissue at the surgical site within the aperture area and to reduce movement of the tissue relative to the platform during the procedure by capturing the tissue adjacent to the surgical site. The connecting cord, thread or tape also aids in the compression of the artery in a grafting procedure to occlude flow through the artery adjacent to the surgical site. The tape is releasably attached to the holder on the outer or inner sides of the platform. A preferred embodiment of the holder can include a plurality of slits or openings positioned on both sides of the stabilizer that receive and frictionally secure the tape on both sides of the aperture. In a further preferred embodiment, the slits or openings may be located along the external surface of the retaining element and may be spaced apart laterally from the aperture area.

A preferred embodiment of the invention comprises a retaining element or base that is open to form an aperture area to permit removal of the stabilizer from around the grafted artery. The stabilizer can be beneficial in any procedure where it is desirable to stabilize a surgical site. For example, the stabilizer can also be used for grafting vessels onto the diagonal, right or other coronary arteries without altering the heart's pumping function.

The coronary arteries are typically about 1-2 mm in diameter, and the pumping heart can move these arteries over distances of several millimeters during each heartbeat. Because the movement of even 1 or 2 millimeters can result in a displacement of the grafting site that can substantially interfere with suturing an effective anastomosis, it is desirable to restrain movement of the artery at the surgical site in any direction to less than about 1 mm. The stabilizer and tapes of the present invention preferably restrain movement of the tissue relative to the base.

In a preferred embodiment of the invention, the handle or articulating arm that is secured to the platform of the stabilizer can be held in position by the user, attached to various locations on a retractor frame that is fixed around the operative site or simply clipped to a drape around the site.

In yet another preferred embodiment of the present invention, the stabilizer includes a generally rectangular, oblong or similar shape wherein the width dimension of the stabilizer is greater than the lengthwise dimension. In the preferred form of the present invention, the stabilizer is generally rectangular and the connector for attachment to a handle or other member is located on the stabilizer generally along the width dimension of the stabilizer such that the application of force along the handle is applied near the aperture area. In a further preferred embodiment, the surgical retractor has tape holder elements disposed in the longitudinal dimension and/or along the periphery of the stabilizer. Each holder element includes one or more slots that frictionally grip an end of a connector such as elastic tape or thread that may be positioned to extend through the aperture to attach tissues to the stabilizer. The use of slots or grooves on the surface of the stabilizer allows the user to quickly place the connectors such as elastic tape or thread around the inner or outer surface of the stabilizer to position tissue at the surgical site within the stabilizer aperture and to minimize movement of the tissue relative to the stabilizer during the procedure. When these slots are used, the tapes are threaded through the tissue of the heart-wall of the patient and then aligned and drawn towards each other to be positioned in the desired slots. The surgeon can include additional tissue around the blood vessel as the tapes are tightened so that the blood vessel is compressed by the adjacent tissue rather than being constricted by the tapes. Additionally, the surgeon can position the tapes at a relatively wider angle of approach when the tapes are threaded around the outer surface of the stabilizer so that more tissue is positioned between the tapes and the blood vessel than when the tapes are drawn through the inner surface of the stabilizer. The route used by the surgeon varies depending on the depth of the desired blood vessel and the surgeon's preferred approach to performing the anastomosis. The combination of the stabilizer and the tapes provides a system that does not require the significant compression or upward traction as required by the prior devices to obtain a suitable surgical site.

In a preferred embodiment, the stabilizer may also include a plurality of tabs extending into the aperture area. The use of the tabs in combination with the tapes causes the tissue in the aperture area to be raised upwardly a small amount to expose the portion of the blood vessel that is to be the subject of the anastomosis while providing a relatively low profile to minimize interference with the surgeon's view and any other instruments used during the procedure. Additionally, the tabs preferably extend into the aperture area to create contact surfaces along three sides of the desired surgical site to further restrict the flow of blood through the blood vessel.

When the present invention is used in a coronary artery bypass procedure, the stabilizer is positioned in a desired position along the myocardial surface of the patient. A tape, for example, silastic tape (i.e. a silicon elastomer) or suture thread, is passed through the myocardium at a location adjacent to the artery grafting site with a blunt needle. Both ends of the tape are connected to the stabilizer platform with sufficient tension to draw the tissue into contact with the bottom surface of the platform and to occlude blood flow on the upstream side of the operative site. The tape compresses the artery against the bottom surface of the platform and preferably against the tabs while the artery graft site is held in a fixed position relative to the aperture area. The stabilizer is then positioned in the desired relative position along the myocardium of the patient. The coronary artery is opened longitudinally and the end of the mammary artery is sewn to the graft opening with multiple fine sutures. The tape is released, blood flow is restored and the anastomosis is then inspected for hemostasis and other defects.

### BRIEF DESCRIPTION OF THE DRAWINGS

FIG. 1 is a top perspective view of a stabilizer in accordance with the embodiment of the invention;

FIG. 2 is a top view of the stabilizer in accordance with the preferred of the invention;

FIG. 3 is an end view of the stabilizer in accordance with the preferred embodiment of the invention showing the tab members in the aperture area an schematically illustrating the compression of a blood vessel;

FIG. 4 is an end view of the stabilizer in accordance with the preferred embodiment of the invention showing the end wall of the stabilizer;

FIG. 5 is a side view of the stabilizer in accordance with the preferred embodiment of the invention showing the tape holders; and

FIG. 6 is a perspective view of a chest retractor and handle supporting a stabilizer in accordance with the present invention.

The foregoing objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

### DETAILED DESCRIPTION OF THE INVENTION

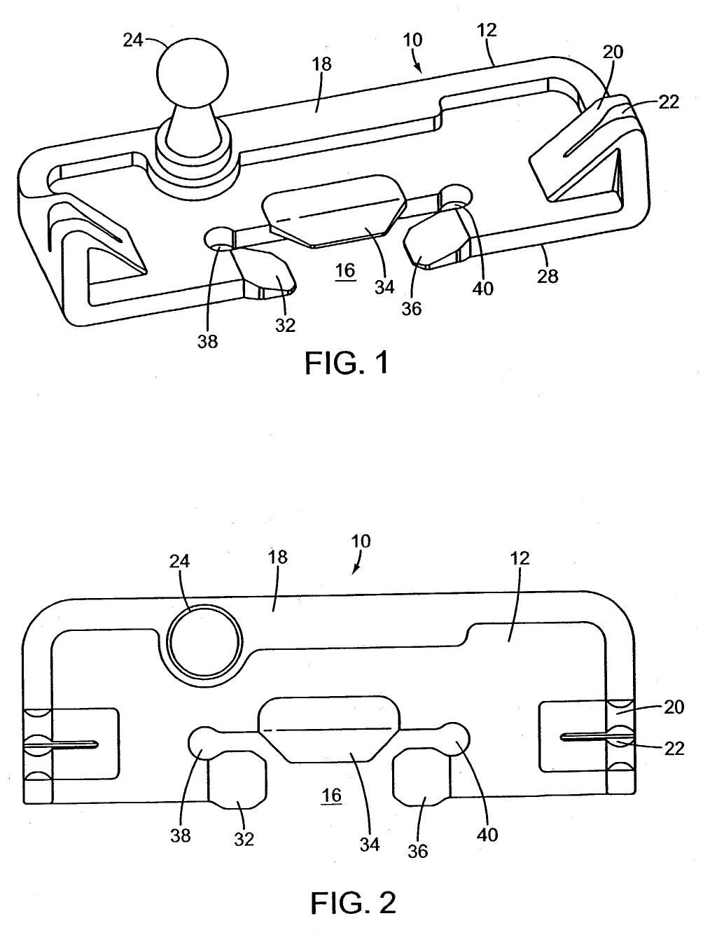

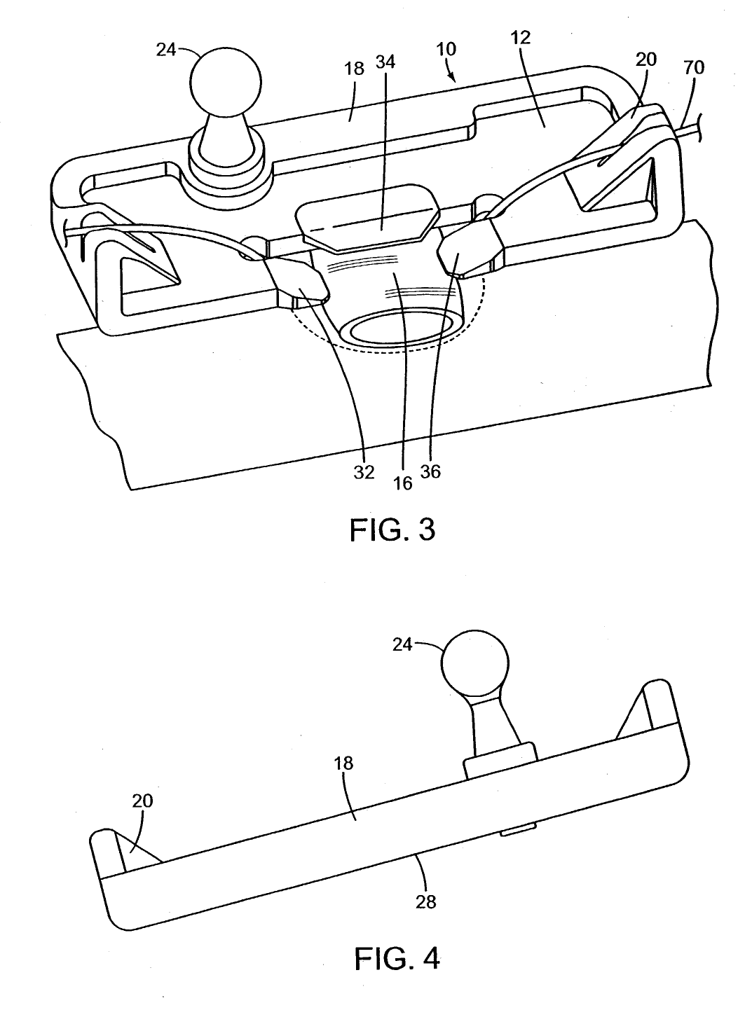

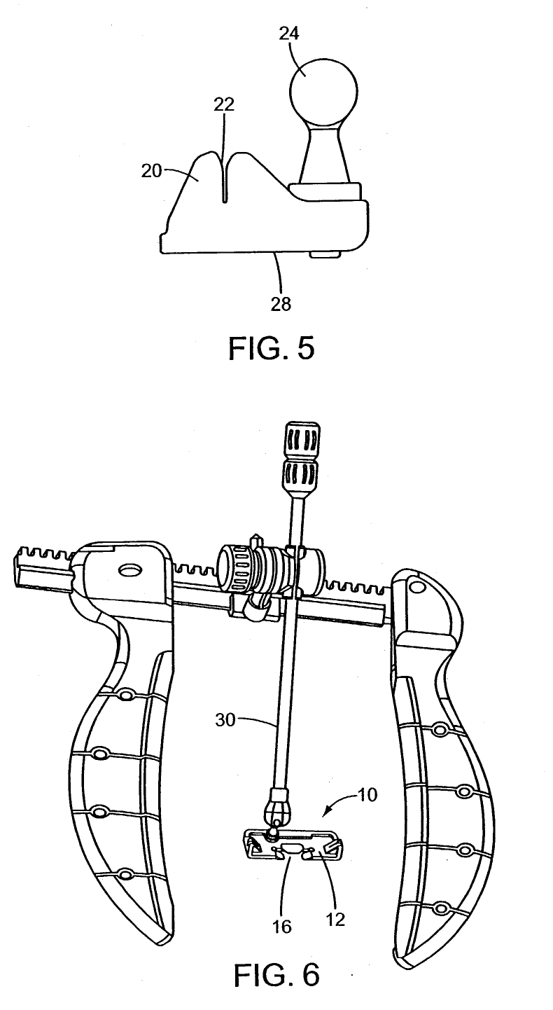

A preferred embodiment of the invention is illustrated in connection with FIG. 1. A stabilizer 10 includes a platform or base 12 having an aperture area 16 that is positioned to expose tissue at a surgical site. The base 12 can be made with a metal or a molded plastic material. The stabilizer 10 can be sterilized after each use, or alternatively, can be disposable after one procedure. A handle 30 or articulating arm can be permanently attached to a connector 24 on the base 12 of the stabilizer 10, or as described below in connection with other preferred embodiments, can be detachable.

In a preferred embodiment of the invention, the platform has a substantially Rectangular, oblong or similar shape with each side having a width in the range between about 0.75 cm. and 2 cm. and a length in the range of about 3.0 cm. and 6 cm. Thus the surface area of the platform is between about 2.25 cm2 and 12 cm2, preferably between about 5 cm2 and 10 cm2. This size fits readily in the incision in the chest of the patient either during typical open chest surgery or less or minimally invasive surgery and can be positioned along nearly any surface of the heart. The exterior surface of the stabilizer 10 preferably includes a raised lip area 18 surrounding three of the four sides of the stabilizer 10. A connector 24, such as a ball member, is positioned along the side and offset from the center of the stabilizer 10. In the preferred form of this invention, the connector 24 is positioned adjacent to the lip area 18 and is attachable to a handle member 30. The remaining side of the stabilizer 10 includes the aperture area 16 and tab members 32, 34 and 36 extending therein. The tab members 32, 34 and 36 are preferably shaped as tapered members that taper gradually from the top surface of the stabilizer inwardly to the bottom surface 28 of the stabilizer 10. Additionally, the aperture area 16 preferably includes a pair of recessed areas 38 and 40 on the sides thereof. The recessed areas 38 and 40 preferably extend laterally from the aperture area 16 and are sized to removably receive the connecting tapes 70 therein. The recessed areas are also preferably aligned with the holder mechanism 20 and slots 22 located along the exterior surface of the stabilizer 10.

The size of the aperture area can be in the range of about 0.1 and 2 cm. in length and about 0.5 and 5 mm in width. The aperture area can be of various configurations including, narrower in the center and wider at the end adjacent to the platform portion of the stabilizer to accommodate the openings or recessed areas 38 and 40. As shown, the depth of the aperture area 16 is preferably less than the lengthwise dimension of the base 12 adjacent to the aperture area 16. The sidewall section of the aperture includes tab members 32 and 36 extending along opposite sides of the aperture area 16. The tape 70 compresses portions of the tissue on the opposite sides of surgical site against the tab members 32 and 36. As shown, the tab members 32 and 36 preferably include a generally octagonal shape having sides that extend outwardly from the base 12 and inwardly into the recessed areas 38 and 40 to assist in the positioning of the tape 70 and the compression of the tissue. The portions of the bottom surface 28 adjacent to the aperture area 16 of the stabilizer are in contact with artery and compress it against the stabilizer generally along the tab member 34. The bottom surface 28 of the base 12 that surrounds the artery and is in contact with the myocardium. may be roughened or abraded to frictionally engage the heart wall around the artery and thereby locally restrict heart motion around the surgical site.

A preferred embodiment of the invention can be used at a surgical site to perform an anastomosis during a bypass procedure. In this particular procedure for a coronary graft without cardiopulmonary bypass, a proximal portion of the LIMA is dissected from the chest wall to expose an end to be grafted onto a grafting site on the coronary artery. Blood flow in the vessel can be occluded with a clamp. In this example, a connector such as a cord or silastic tape 70 is threaded through myocardium. surface under the coronary artery on opposite sides of the grafting site. The exposed surface of heart is undergoing substantial three dimensional movement during the procedure as the heart is allowed to continue beating in the usual manner. Alternately, a saphenous vein or similar blood vessel from the patient may be used to provide the bypass conduit for the coronary artery of the patient. The stabilizer 10 preferably serves to immobilize the grafting site using the platform portion of the stabilizer and the connecting tape 70 which is stretched and attached to a holder mechanism 20 including one or more slots 22 in the peripheral edge of base 12. As described in greater detail below, the ends of the tapes 70 can be manually positioned in the slots 22 to allow the user to adjust the tension in the tapes or threads.

The aperture area 16 is oriented on the stabilizer to extend longitudinally along the axis of artery. The tape 70 exerts a compressive force on the artery to press the artery against a bottom surface 28 of the stabilizer 10. More particularly, the tape 70 extends in a direction that is substantially perpendicular to the artery axis exposed in the aperture area 16. The aperture area can have a first pair of lateral sections or recessed areas 38 and 40 that are aligned to accommodate the positioning of the ends of the tape 70. The recessed areas 38 and 40 preferably extend laterally from the sides of the aperture area 16 to enable the user to move the tape out of the operative field while tape 70 draws the tissue upwardly into the aperture area. Alternatively, holes (not shown) extending through the base 12 that are separated from the aperture can be used. The holes may be large enough to provide easy tape 70 feed through and can be angled towards the bottom center to provide compression of the artery at lower tension of the tape.

In the preferred embodiment of the present invention, the stabilizer 10 can have a connector 24 or handle attachment post so that the user can attach a handle 30 to provide convenient access to the aperture area 16 and facilitate immobilization of the artery and surrounding tissue. The handle 30 can be positioned between or adjacent to the tapes relative to the aperture area and preferably extending above the top surface of the stabilizer 10. This structure exerts little downward force or upward force on the heart on the artery while immobilizing the artery at the surgical site. Also the anterior-posterior compression of the artery avoids trauma to the artery due to circumferential compression of the artery and adjacent tissue. As shown, the tape 70 under the bottom surface of the tabs 32 and 36 lifts the artery to form an occlusion by compressing the artery between the tissue captured by the tape and the bottom surface of the stabilizer 10.

The tape is inserted in the myocardium with a blunt needle approximately 1-2 cm. apart and the stabilizer is positioned with the tape being pulled through the aperture area and positioned in the recessed areas 38 and 40 thereof. The tape is connected to the slots 22 of the stabilizer 10 to compress the artery and occlude blood flow distally or upstream of the grafting site. The tension in the tapes can optionally be adjusted during the procedure to minimize blood loss at the site. After the procedure is complete, the stabilizer 10 may be easily removed from the surgical site. In the preferred embodiment, the base 12 includes the open ended aperture area 16 to enable the completed anastomosis to be removed by releasing the tapes to reopen the artery and then the anastomosis may be easily removed therefrom once the tapes are released from the slots 22.

The stabilizer is secured at the site by attaching the stabilizer to the handle or arm and to the chest retractor or other implement. The grafting site preferably undergoes a minimal amount of movement relative to the stabilizer in any direction during this example procedure. Additionally, the site may be suctioned or irrigated during performance of the anastomosis. Thereafter, the grafting site is inspected, the tapes are released from the holders, and the retractor is removed. After blood flow is restored, the site is inspected and closed.

Although the use of the stabilizer has been described in connection with a particular bypass procedure, it can also be used in other procedures such as bypass operations involving the diagonal, right or other coronary artery where movement at the site can interfere with the procedure.

Alternative embodiments involve opening of the chest and positioning the stabilizer at any exposed site on the heart wall or surrounding areas to immobilize the operative site. The stabilizer serves to isolate the site and limits or stops motion at the site due to respiratory movement of the lungs or the pumping motion of the heart.

In the preferred embodiment, a retractor system or frame manufactured by Genzyme Corporation is illustrated in FIG. 6 to support a stabilizer in accordance with the preferred form of the present invention.

While this invention has been particularly shown and described with references to preferred embodiments thereof, it will be understood by those skilled in the art that various changes in form and details may be made therein without departing from the spirit and scope of the invention as set forth in the appended claims.

What is claimed:

1. A surgical device for a coronary bypass procedure comprising:
a stabilizer having a platform area having a width dimension that is greater than the longitudinal dimension and an aperture area defining an operative site;
a plurality of holders on the stabilizer; and
a connector that extends underneath an artery such that the connector compresses the artery against a surface on the stabilizer at a first arterial compressing position on a first side of the operative site and the holders are positioned to receive the connector therein.

2. The surgical retractor of claim 1, having raised sidewall elements along the longitudinal dimension of the stabilizer.

3. The surgical retractor of claim 2, wherein the raised sidewall elements have at least one slot therein to frictionally grip an end of the connector.

4. The surgical retractor of claim 1, wherein the connector comprises flexible tape or thread.

5. The surgical retractor of claim 1, wherein the stabilizer comprises a first, a second, a third and a fourth side and at least one side has a tab member adjacent to the aperture area and a gradually thinner cross-section to provide a downward slope toward the aperture area.

6. The surgical retractor of claim 1, wherein the stabilizer has an aperture area having a plurality of recessed areas extending therefrom to allow for the placement of the connector under the stabilizer and through the recessed areas to position tissue at the surgical site within the aperture area.

7. The surgical retractor of claim 1, wherein the stabilizer has a plurality of tab members adjacent to the aperture area.

8. The surgical retractor of claim 1, wherein the stabilizer comprises a first, a second, a third and a fourth side and a plurality of said sides have a raised area extending therealong.

9. The surgical retractor of claim 1, wherein the stabilizer includes a raised connector thereon for the attachment of the stabilizer to a handle member.

10. The surgical retractor of claim 9 wherein said raised connector is positioned adjacent to a side of the stabilizer and generally along the width dimension thereof.

11. A surgical retractor comprising:
A rigid retaining element having a generally rectangular platform section wherein said platform section has a width dimension that is at least twice as large as the lengthwise dimension and wherein said lengthwise dimension is formed by a pair of laterally extending members and said width dimension and said laterally extending members from an aperture area therebetween and wherein said aperture area is sized to allow the formation of a graft at a surgical site therein;
said aperture area including a plurality of recessed areas extending laterally therefrom a pair of holder members on said platform section and wherein said holder members are aligned with said recessed areas of said aperture area.

12. The surgical retractor of claim 11 further including a raised connector and said raised connector is sized for attachment to a handle member to allow for the application of a tissue restraining force against the surgical site by said stabilizer.

13. The surgical retractor of claim II further including a flexible connector removably attached to said holder members wherein said flexible connector is interconnected with said stabilizer to allow for the application of a tissue restraining force against the surgical site adjacent to said aperture area.

14. A method of stabilizing a surgical site during surgery comprising the steps of:
positioning a stabilizer at the surgical site wherein the stabilizer is formed having a width dimension more than twice the lengthwise dimension thereof and wherein the lengthwise dimension is formed of a pair of laterally extending members and said width dimension and said laterally extending members form an aperture area therebetween and wherein the aperture area is sized to allow the formation of a graft therein;
threading a flexible connector through tissue surrounding the surgical site and removably attaching the flexible connector to the stabilizer to stabilize the tissue adjacent to the surgical site with respect to the stabilizer;
performing a surgical procedure; and
releasing the flexible connector from the stabilizer and removing the stabilizer from around the graft performed in the aperture area.

15. The method of claim 14 further including the step of releasably attaching the flexible connector to a plurality of holding members on the stabilizer to apply an upwardly directed force to the tissue adjacent to the surgical site.

16. A method of stabilizing a surgical site during surgery comprising the steps of:
positioning a stabilizer at the surgical site wherein the stabilizer is formed having width dimension more than twice the lengthwise dimension thereof and wherein the lengthwise dimension is formed of a pair of laterally extending members and said width dimension and said laterally extending members form an aperture area therebetween along three sides of the surgical site;
attaching a handle member to the stabilizer to stabilize the tissue at the surgical site with respect to the stabilizer:
performing a surgical procedure; and
moving the stabilizer from around the graft performed in the aperture area.

17. The method of claim 16 further including the step of threading a flexible connector through tissue surrounding the surgical site and removably attaching the flexible connector to the stabilizer to stabilize the tissue adjacent to the surgical site with respect to the stabilizer.

18. A method of stabilizing a surgical site during surgery comprising the steps of:
positioning a stabilizer at the surgical site wherein the stabilizer is formed having a width dimension more than twice the lengthwise dimension thereof and wherein the lengthwise dimension is formed of a pair of laterally extending members and said width dimension and said laterally extending members form an aperture area therebetween;
attaching a handle member to the stabilizer to stabilize the surgical site with respect to the stabilizer to apply a downward retaining force to the surgical site with the stabilizer: attaching a flexible connector to the stabilizer to stabilize the tissue adjacent to the surgical site with respect to the stabilizer to apply an upward retaining force to the surgical site: performing a surgical procedure; and
removing the stabilizer from around the graft performed in the aperture.
